(19) United States
(12) Patent Application Publication
(10) Pub. No.: US 2012/0064513 A1
(43) Pub. Date: Mar. 15, 2012

(54) Cell Sensor, And Monitoring Method Using Same For The Real-Time Monitoring Of Cell Capacitance

(75) Inventors: Kyung Hwa Yoo, Seoul (KR); Chae Ok Yun, Seoul (KR); Dong Hyun Kim, Seoul (KR); Ri Mi Lee, Daegu (KR); Kyu Jung Kim, Seoul (KR); Pyung Hwan Kim, Seoul (KR)

(73) Assignee: Industry-Acaddemic Cooperation Foundation, Yonsei University, of Seoul, Seoul (KR)

(21) Appl. No.: 13/264,096

(22) PCT Filed: Apr. 7, 2010
(86) PCT No.: PCT/KR10/02143

§ 371 Date: Oct. 12, 2011

(30) Foreign Application Priority Data
Apr. 10, 2009 (KR) 10-2009-0031298

(51) Int. Cl. C12Q 1/70; G01N 27/22; C12Q 1/02; C12M 1/34

(52) U.S. Cl. 435/5; 435/287.1; 435/287.2; 435/7.21; 435/29; 435/7.23

(57) ABSTRACT

The present invention relates to a cell sensor for real-time monitoring of cell capacitance and a monitoring method using the same, and more particularly, to a cell sensor capable of monitoring an endocytosis process of a biomolecule through a cell surface receptor by attaching a cell between electrodes and then measuring in real time the capacitance between the electrodes over time, and a monitoring method using the same. A cell sensor for the real-time monitoring of cell capacitance according to an exemplary embodiment of the present invention includes a substrate; a first electrode and a second electrode that are formed on the substrate and spaced apart by a gap from each other, in which at least one or more cells are introduced to be attached onto the gap; and a passivation layer that is formed on each of the tops of the first electrode and the second electrode to prevent the cells from being attached onto the top of the first electrode and the second electrode. The cell sensor for the real-time monitoring of cell capacitance and the method for real-time monitoring of the cell condition using the same according to an exemplary embodiment of the present invention have the effect of being able to monitor an endocytosis process of a biomolecule through a cell surface receptor in real time, by attaching cells having a receptor for the particular biomolecule between electrodes and then measuring in real time the capacitance between the electrodes over time.

### TECHNICAL FIELD

The present invention relates to a cell sensor for real-time monitoring of cell capacitance and a monitoring method using the same, and more particularly, to a cell sensor capable of monitoring an endocytosis process of a biomolecule through a cell surface receptor by attaching a cell between electrodes and measuring in real time the capacitance between the electrodes over time, and a monitoring method using the same.

### BACKGROUND ART

Adsorption of biomolecules onto target cell receptors includes an antigen-antibody reaction which specifically occurs between antibodies and corresponding antigens. Because of high specificity, the antigen-antibody reaction does not occur if there are small changes in any one of the antibodies and antigens.

For another example, a virus generally binds to a cell surface receptor (protein or sugar) to initiate cell infection. Specifically, when adenovirus binds to a coxsackie/adenovirus receptor (CAR) which is a crucial receptor for adenovirus, an interaction between the virus and receptor occurs, and thus the virus enters the cells via clathrin-mediated endocytosis.

To detect the internalization process of biomolecules, the biomolecules are generally labeled with fluorescence and observed under a fluorescence microscope. However, a long time (24 hrs or longer) is required for fluorescence expression within the virus, and thus it is difficult to immediately detect the adsorption of biomolecules. The method is also disadvantageous in that low production efficiency of the fluorescent proteins for envelope adsorption of biomolecules could increase costs. Furthermore, passage of the fluorescence through cell membrane cannot be exactly examined under the fluorescence microscope, and thus, it is difficult to monitor activities of the biomolecules in real time.

### DISCLOSURE

### Technical Problem

An object of the present invention is to provide a cell sensor capable of monitoring changes in cell potential in real time by attaching one or more cells onto a gap area between electrodes of the cell sensor, and a method for real-time monitoring of changes in cell potential using the same by measurement of changes in capacitance during a adsorption and endocytosis process, which is a specific binding process between a cell surface receptor and a biomolecule or a virus.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

### Technical Solution

A cell sensor for real-time monitoring of cell capacitance according to an exemplary embodiment of the present invention includes a substrate; a first electrode and a second electrode that are formed on the substrate and spaced apart by a gap from each other, in which at least one or more cells are introduced to be attached onto the gap; and a passivation layer that is formed on each of the tops of the first electrode and the second electrode to prevent the cells from being attached onto the top of the first electrode and the second electrode.

The cell sensor is able to monitor cell condition in real time by measuring capacitance or impedance between the first electrode and the second electrode.

Further, the cell sensor may further include a fluidic channel that is formed to have the gap area and includes an inlet to introduce at least one of the biomolecules, which undergo an antigen-antibody reaction with the cells, and the cell death-inducing agents into the gap area and an outlet to extract at least one of biomolecules and cell death-inducing agents from the gap area; and the fluidic channel includes at least one of acryl and PDMS (Polydimethylsiloxane), and is attachable and detachable from the substrate and the first and second electrodes.

In addition, the cell sensor may further include a well to prevent a cell culture medium from leaking out of the gap area where the cells are introduced; and the well may include at least one of acryl and PDMS (Polydimethylsiloxane).

Moreover, the cell sensor may include a plurality of electrode pairs composed of the first and second electrodes, which are arrayed on the substrate in common.

A method for monitoring a cell condition in real time according to another exemplary embodiment of the present invention includes the steps of (a) introducing the cells into the gap area of the aforementioned cell sensor and then introducing a cell culture medium for cell cultivation thereto; (b) introducing biomolecules capable of specifically binding with the cells into the cells cultured in the cell culture medium; and (c) measuring capacitance between the first electrode and the second electrode in real time during cultivation of the cells of step (b).

In this regard, the cells may be any one of HEP1 cell highly expressing CAR (coxsackie and adenovirus receptor) which is a target receptor of adenovirus and CAR-antibody, and 435 breast cancer cell overexpressing ErbB-2 (HER2/neu) which is a target receptor of herceptin, the cell culture medium includes at least one of DMEM (Dulbecco's Modified Eagle Medium), RPMI (Roswell Park Memorial Institute) 1640 Medium, and MEM (Minimum Essential Medium), and the biomolecule may be any one of adenovirus, CAR-antibody, and herceptin.

In step (c), endocytosis of the biomolecules into the cells through the cell surface receptor can be monitored in real time by real-time monitoring of capacitance, and the step of controlling the concentration of the biomolecule using the cell culture medium may be further included, prior to step (c).

A method for monitoring the cell condition in real time according to yet another exemplary embodiment of the present invention includes the steps of (a) introducing the cells into the gap area of the aforementioned cell sensor and then introducing a cell culture medium for cell cultivation thereto; (b) introducing a cell death-inducing agent into the cells cultured in the cell culture medium; and (c) measuring capacitance between the first and second electrodes in real time during cultivation of the cells of step (b).

In this regard, the cell death-inducing agent may include any one of viruses, bacteria, nucleic acids, drugs and metal particles that contain TRAIL (Tumor necrosis factor Related Apoptosis Inducing Ligand).

In step (c), endocytosis of the biomolecules into the cells through the cell surface receptor or adsorption can be monitored in real time by real-time monitoring of capacitance and impedance.

A method for monitoring cell condition in real time according to still another exemplary embodiment of the present invention includes the steps of (a) introducing the cells into the gap area of the aforementioned cell sensor and then introducing a cell culture medium for cell cultivation thereto; (b) introducing a biomolecule together with a cell death-inducing agent into the cells cultured in the cell culture medium; and (c) measuring capacitance between the first and second electrodes in real time during cultivation of the cells of step (b).

### Advantageous Effects

In accordance with a cell sensor for real-time monitoring of cell capacitance and a method for real-time monitoring of a cell condition using the same according to an exemplary embodiment of the present invention, an endocytosis process of a biomolecule through a cell surface receptor can be monitored in real time by attaching at least one or more cells having a receptor for the particular biomolecule between electrodes and then performing a real-time measurement of the capacitance between the electrodes over time.

### DESCRIPTION OF DRAWINGS

FIGS. 1 to 4 are views showing an example of a cell sensor for real-time monitoring of cell capacitance according to the present invention.

FIGS. 5 to 7 are views illustrating an example of a plurality of electrode pairs arrayed on one substrate.

FIG. 8 is a view of illustrating the measurement equipment including an incubator for cell culture in the cell sensor.

FIG. 9 is the result of real-time monitoring of cell capacitance during HEP-1 cell growth and death by a cell death-inducing agent, TRAIL (TNF related apoptosis inducing ligand).

FIG. 10 is the result of real-time monitoring of cell capacitance when the cells are treated with Ad-Δ19 virus, dl-ΔE1 virus, and dl-ΔE1/ΔE3 virus at a concentration of 1*10⁵ per chamber.

FIG. 11 is a view of illustrating the process of endocytosis.

FIG. 12 is the result of real-time monitoring of cell capacitance when the cells are treated with (a) Ad-Δ19 virus, (b) dl-ΔE1 virus, and (c) dl-ΔE1/ΔE3 virus using a CAR-antibody, a protein inhibitor, and Ribavirin.

FIG. 13(a) is the result of real-time monitoring of cell capacitance when HEP1 cells are treated with Δ19 virus and CAR-antibody, and FIG. 13(b) is the result of real-time monitoring of cell capacitance when HEP1 cells are treated with Δ19 virus and CAR-antibody, and 435 breast cancer cells overexpressing ErbB-2 (HER2/neu) which is a target receptor of herceptin are treated with herceptin, for comparison therebetween.

FIG. 14 is the result of real-time monitoring of cell capacitance when 435 breast cancer cells are treated with different concentrations of herceptin.

FIG. 15 is the result of real-time monitoring of cell capacitance when endocytosis by adsorption occurs.

FIG. 16 is the result of real-time monitoring of cell capacitance when the cells are treated with different concentrations of polystyrene beads.

FIG. 17 is the result of real-time monitoring of cell capacitance when the cells are treated with different concentrations of gold nanoparticles.

FIG. 18 is TIRF (Total Internal Reflection Fluorescence) microscope images showing the endocytosis of GFP-tagged virus via the receptor on the cell.

FIG. 19 is TIRF (Total Internal Reflection Fluorescence) microscope images showing endocytosis of rhodamine (red dye)-loaded polystyrene bead by adsorption.

### BEST MODE

Hereafter, exemplary embodiments of the present invention will be described in detail with reference to the accompanying drawings.

First of all, it is to be noted that in giving reference numerals to elements of each drawing, like reference numerals refer to like elements even though like elements are shown in different drawings. Further, when it is determined that the detailed description related to a known configuration or function may render the purpose of the present invention unnecessarily ambiguous in describing the present invention, the detailed description will be omitted here. Further, the preferred embodiments of the present invention will be described hereinbelow, but it will be apparent to those skilled in the art that various modifications and changes may be made thereto without departing from the scope and spirit of the invention.

The cell sensor for real-time monitoring of cell capacitance according to an exemplary embodiment of the present invention is used for real-time monitoring of an endocytosis process and adsorption of biomolecules such as a virus and an antibody, which specifically bind with cell surface receptors, that is, a virus receptor or an antigen.

More specifically, the cell sensor for real-time monitoring of cell capacitance is used for real-time monitoring of endocytosis of biomolecules through cell surface receptors by performing real-time measurement of the capacitance between electrodes over time after attaching a cell having a receptor of a particular biomolecule onto the gap area between the electrodes. Identification of a viral infection, Biomolecular Screening or the like can be performed by real-time monitoring.

Hereinbelow, a cell sensor will be first described, and then a method for real-time monitoring of a cell condition using the same and experimental results thereof will be described in detail.

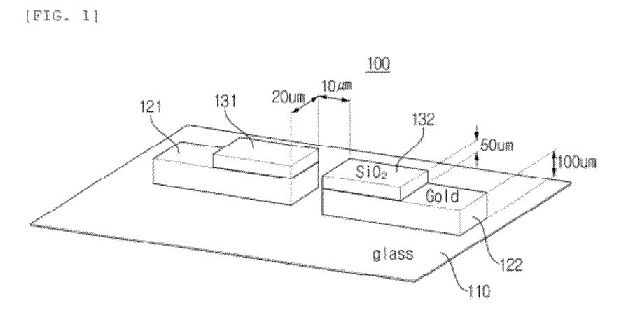

FIGS. 1 to 4 are views showing an example of a cell sensor for real-time monitoring of cell capacitance according to the present invention.

As shown in FIG. 1, the cell sensor 100 for real-time monitoring of the potential change of a cell 200 may include a substrate 110, a first electrode 121 and a second electrode 122, and passivation layers 131 and 132.

The substrate 110 may include a nonconductor such as glass, and the first electrode 121 and the second electrode 122 are formed on the substrate 110 and spaced apart by a gap from each other, in which the cell 200 are introduced so as to be attached onto the gap area.

The first electrode 121 and the second electrode 122 may be spaced apart by a gap of 8 um (micrometer) to 100 um, and each of the first electrode 121 and the second electrode 122 may be made to have a height of 80 nm (nanometer) to 200 nm and a width of 18 um to 100 um, and their material may be any one of electrically conductive gold, platinum, and conductive polymers, and other electrically conductive materials may be used.

In FIG. 1, for example, the first electrode 121 and the second electrode 122 were spaced apart by a gap of 10 um, each of them had a height of 100 nm and a width of 20 um, and they were made of a gold-containing material.

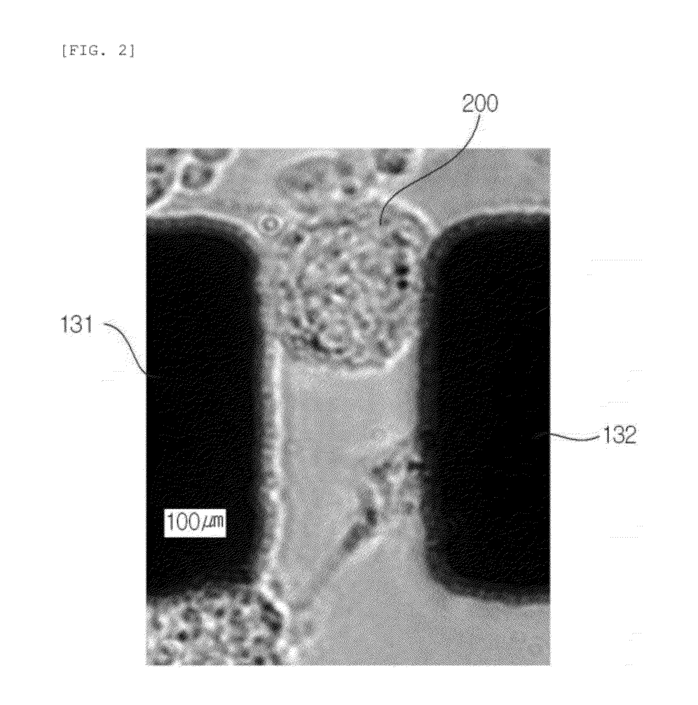

In this regard, the first electrode 121 and the second electrode 122 are spaced apart by a gap of 8 um (micrometer) to 12 um so as to attach a single cell 200 onto the gap area by making the gap size similar to the typical size of a single cell 200 as shown in FIG. 2.

In FIG. 1, the gap of 10 um between the first electrode 121 and the second electrode 122 is illustrated, but the first electrode 121 and the second electrode 122 are made to have a gap of 8 um (micrometer) to 100 um therebetween so as to monitor a plurality of cells at the same time. As such, the first electrode 121 and the second electrode 122 can be made to have a variety of gap sizes. Hereinbelow, a cell sensor with the gap of 10 um between the first electrode 121 and the second electrode 122 will be described.

As such, at least one or more cells 200 are attached onto the gap area between a pair of electrodes, thereby performing real-time monitoring of the potential changes of the cell membrane 200 upon endocytosis of a biomolecule into the cell 200 through a target receptor, and also performing real-time monitoring of the potential changes associated with the cytoplasm 200 and protein synthesis within the cell 200 caused by endocytosis of the biomolecule.

More particularly, in the cell sensor, the electrodes measuring the cell 200 function as a capacitor by culturing the cells 200 placed between a pair of electrodes. In the cell 200, the cell membrane 200 composed of a lipid bilayer separates the interior of the cell 200, where a lot of charged molecules are present, from the outside media, and thus it provides an insulating function.

Because of the presence of the cell membrane 200, the cell 200 behaves as a non-conductor, but bipolarity is induced in the cell 200 when exposed to an AC electric field in the media. In addition, dielectric property of the cell 200 is affected by size and shape of the cell 200, surface charge of the cell membrane 200, conductivity inside the cell 200 or the like, and the dielectric property can be determined by measurement of the capacitance at a low frequency (3 kHz).

Using the same, real-time monitoring of the potential changes of the cell membrane 200 can be performed upon endocytosis of a biomolecule into the cell 200 through a target receptor, and real-time monitoring of the potential changes associated with the cytoplasm 200 and protein synthesis within the cell 200 caused by endocytosis of the biomolecule, or the conductivity changes inside the cell 200 can also be performed by measuring capacitance changes.

The passivation layers 131 and 132 are formed on each of the tops of the first electrode 121 and the second electrode 122 to prevent the cell 200 from being attached onto the top of the first electrode 121 and the second electrode 122.

The passivation layers 131 and 132 may be spaced apart by a gap of 8 um to 100 um, and each of the passivation layers 131 and 132 may be made to have a height of 45 nm to 55 nm and a width of 18 um to 100 um. In order to prevent the cell 200 from being attached onto the top of the first electrode 121 and the second electrode 12, the passivation layers 131 and 132 may be made of non-conductive materials, and may include at least one of PMMA (Polymethyl Methacrylate) and silicon oxide (SiO₂). The passivation layers formed on the top of the first electrode and the second electrode can be made to have a variety of gap sizes, heights, and widths depending on the gap size, height, and width of the electrodes.

As an example of the passivation layers 131 and 132, FIG. 1 illustrates the passivation layers 131 and 132 that have a gap of 100 um, a height of 50 nm and a width of 20 um, and are made of silicon oxide (SiO₂), respectively.

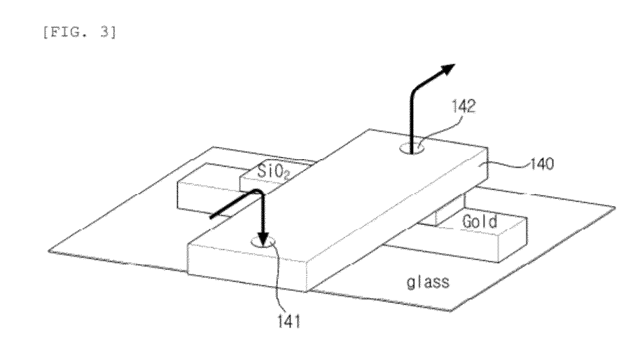

The cell sensor 100 for real-time monitoring of potential changes of the cell 200 may further include a fluidic channel 140, as shown in FIG. 3.

The fluidic channel 140 is formed to have the gap area between the first electrode 121 and the second electrode 122, and includes an inlet 141 to introduce at least one of biomolecules, which undergo an antigen-antibody reaction with the cell 200, and cell death-inducing agents into the gap area and an outlet 142 to extract at least one of the biomolecules and the cell death-inducing agents from the gap area. The fluidic channel 140 maintains transparency until it has a predetermined thickness, and may include at least one of acryl and PDMS (Polydimethylsiloxane) for durability. The fluidic channel 140 may be designed to be attachable and detachable to and from the substrate 110 and the first and second electrodes in order to introduce the biomolecule or cell death-inducing agent after washing and sterilizing a pair of electrodes of the cell sensor 100 under the removal of fluidic channel 140.

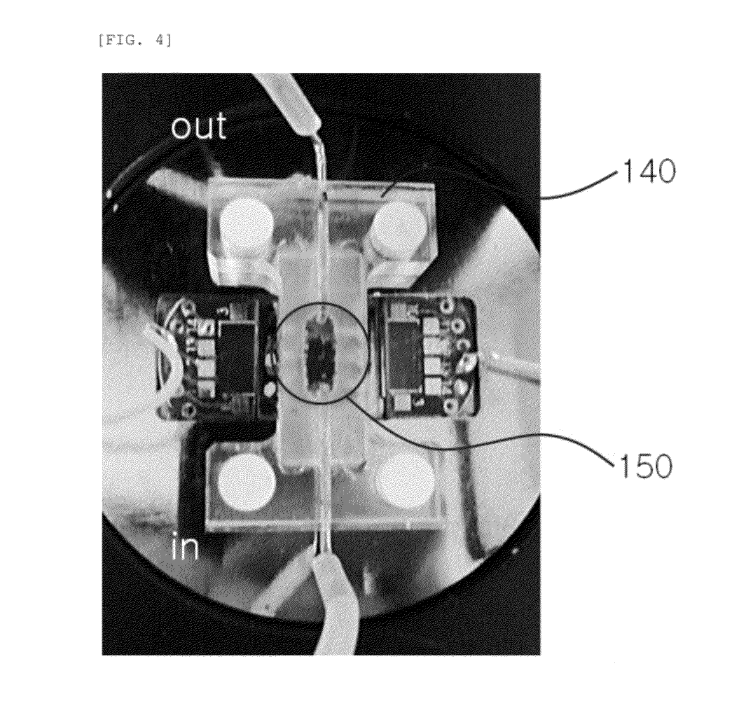

FIG. 4 illustrates an actual view of an exemplary cell sensor 100 for real-time monitoring of potential changes of the cell 200.

As illustrated, the cell sensor 100 may further include a well 150 to prevent a cell culture medium from leaking out of the gap area where the cell 200 is introduced.

The well 150 may be formed inside the fluidic channel 140, and may be formed by including at least one of acryl and PDMS (Polydimethylsiloxane), which is identical to that of the fluidic channel 140. The well 150 may be formed inside the aforementioned fluidic channel 140 in an integrated form.

The cell sensor 100 is connected to an LCR meter to measure capacitance, thereby monitoring potential changes of the cell 200 in real time. At this time, 10 mV of AC voltage at a frequency of 3 KHz is applied to the cell sensor 100. In this regard, the frequency of AC voltage applied to the cell sensor 100 may vary depending on the gap size between the first electrode 121 and the second electrode 122, and any frequency other than 3 KHz is also possible, as long as it is a low frequency.

The cell sensor 100 may include a plurality of electrode pairs composed of the first electrode 121 and the second electrode 122, which are arrayed on the substrate 110 in common.

In addition, a plurality of electrode pairs are made to be arrayed on one substrate 110, so as to measure a plurality of samples at one time and maximize the measurement efficiency.

FIGS. 5 to 7 are views illustrating a plurality of electrode pairs arrayed on one substrate.

Hereinbelow, a cell sensor 100 composed of a pair of electrodes is referred to as “unit cell sensor 100”, and a cell sensor 100 fabricated by arrangement and integration of a plurality of unit cell sensors 100 is referred to as an “array cell sensor”.

In the array cell sensor, a plurality of unit cell sensors 100 are arranged so that different voltages are applied to each electrode pair, and the wiring pattern of each unit cell sensor 100 is formed in one direction to facilitate the measurement of each unit cell sensor 100, as shown in FIG. 5. Each unit cell sensor 100 uses the substrate 110 in common and an integrated well 150 is provided. The well 150 may be easily fabricated using a method such as injection molding or the like, and may be provided to be attachable and detachable.

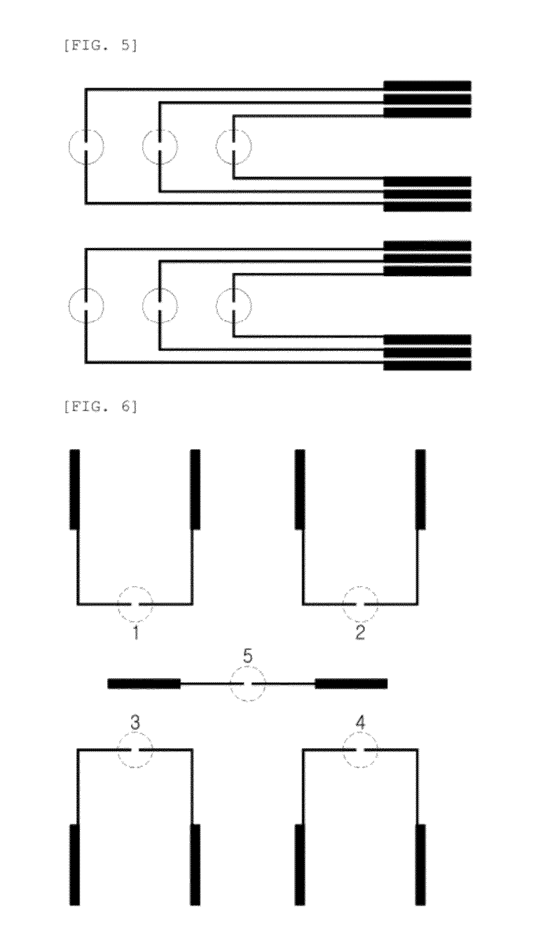

Unlike this, the array cell sensor may be fabricated as shown in FIG. 6.

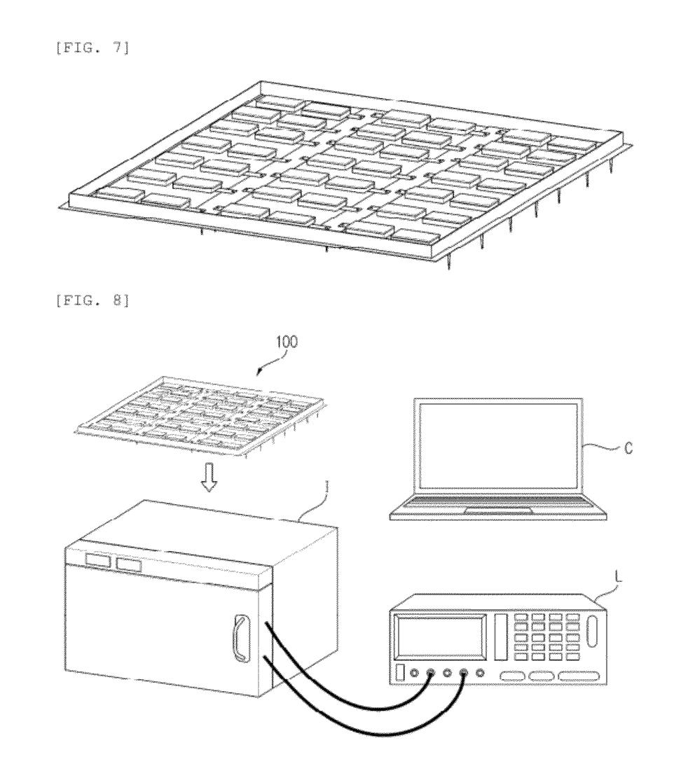

Further, in the array cell sensor, the electrode pairs of a plurality of unit cell sensors 100 may be arranged so that a common voltage is applied thereto, as shown in FIG. 7.

The well 150 formed in each unit cell sensor 100 of the array cell sensor may be formed by photolithography.

The array cell sensor where a plurality of unit cell sensors are integrated on one substrate can be used to monitor a plurality of cells at the same time.

FIG. 8 is a view of illustrating the measurement equipment including an incubator for cell culture in the cell sensor.

The incubator for cell culture (I) maintains optimal temperature and carbon dioxide level for cell culture. Each electrode of the cell sensor 100 is electrically connected to the terminal of the LCR meter (L), and capacitance measured between the electrodes by the LCR meter (L) can be plotted by a computer (C).

A method for monitoring a cell condition in real time using the cell sensor according to the preferred embodiment of the present invention is as follows.

First, the aforementioned cell sensor is fabricated. The fabricated electrode is sterilized using autoclave equipment and ethanol, and then the well made of acryl or PDMS is attached on the electrode for cell culture.

Next, cells grown to a predetermined density are harvested with trypsin-EDTA, and introduced and attached onto the gap area between the electrodes using a pipette.

Subsequently, the cell-loaded electrodes are placed in a 5% CO₂ cell culture incubator at 37° C. and incubated for 12 hrs, in order to tightly attach the cells onto the gap between the electrodes. During incubation, the cells are observed under an optical microscope, whether their conditions are suitable for the experiment.

Thereafter, the fluidic channel is mounted onto the electrodes where the cells are tightly attached, and is fixed on the stage of an optical microscope, of which the bottom is maintained at 37° C. Then, a cell culture media and at least one of biomolecules, cell death-inducing agents and metal particles for observation of endocytosis or adsorption is introduced through the inlet of the fluidic channel. In this regard, the concentration of at least one of the biomolecules and cell death-inducing agents may be controlled using the cell culture media.

During the cell cultivation, capacitance between the first electrode and the second electrode is measured in real time, and thus endocytosis of the biomolecules into the cells through the cell surface receptor or adsorption is monitored in real time.

In this regard, the cells may be any one of HEP1 cell highly expressing a CAR (coxsackie and adenovirus receptor) receptor and 435 breast cancer cell overexpressing an ErbB-2 (HER2/neu) receptor.

In this regard, the cell culture medium may include at least one of 10% FCS (fetal calf serum)-containing media for animal cell culture, DMEM (Dulbecco's Modified Eagle Medium), RPMI (Roswell Park Memorial Institute) 1640 Medium, and MEM (Minimum Essential Medium).

Further, the biomolecule may be any one of adenovirus, CAR antibody (CAR-antibody), and herceptin. In this regard, herceptin is an antibody against ErbB-2 (HER2/neu) and is an anticancer agent used for metastatic breast cancer, osteosarcoma or the like.

In addition, the cell death-inducing agent may include any one of viruses, bacteria, nucleic acids, and drugs that contain TRAIL (Tumor necrosis factor Related Apoptosis Inducing Ligand).

The metal particles may be metal particles of nanometer size, and in the exemplary embodiment of the present invention, a gold nanoparticle with a diameter of 10 nm was used as a metal particle.

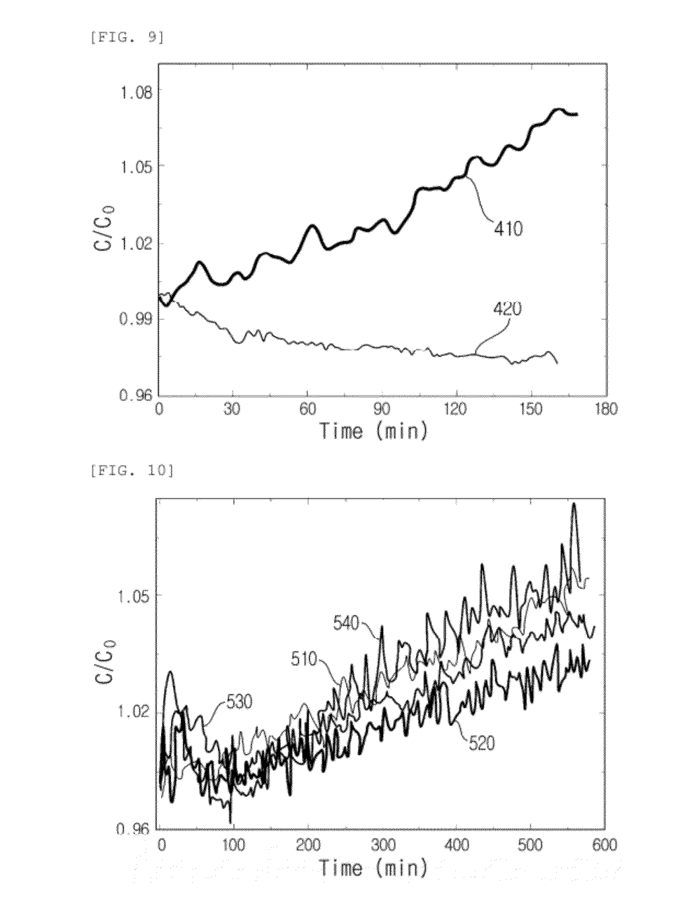

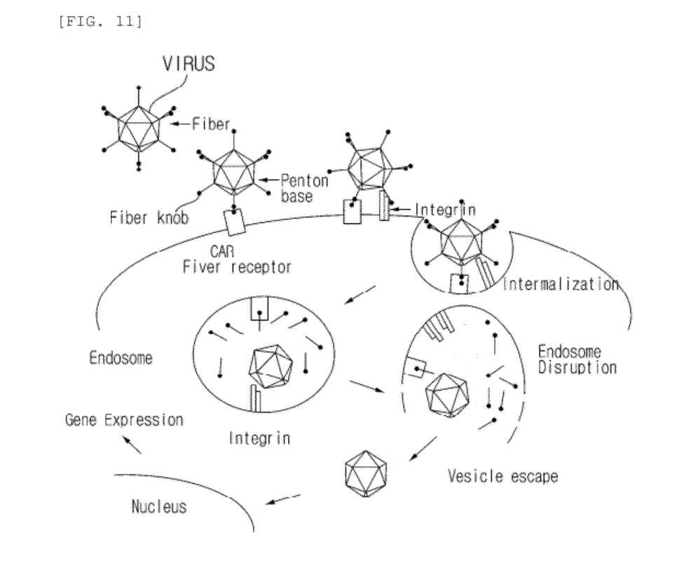

FIGS. 9 to 11 are the results of real-time monitoring of cell capacitance using the cell sensor.

FIG. 9 is the result of real-time monitoring of cell capacitance during HEP-1 cell growth and death by a cell death-inducing agent, TRAIL (TNF related apoptosis inducing ligand). In this regard, TRAIL was made to have a final concentration of 50 ng/ml using the cell culture media.

As illustrated, when the cell capacitance is measured using the cell sensor according to an exemplary embodiment of the present invention, potential energy of the cell increases to increase capacitance during cell growth 410, and on the contrary, capacitance decreases during cell death 420.

FIG. 10 is the result of real-time monitoring of cell capacitance when the cells are treated with Ad-Δ19 virus, dl-ΔE1 virus, and dl-ΔE1/ΔE3 virus at a concentration of 1*10⁵ per chamber.

Herein, 510 represents capacitance change during HEP1 cell growth, 520 represents capacitance change when HEP1 cells are treated with dl-ΔE1 virus at a concentration of 1*10⁵ per chamber, 530 represents capacitance change when HEP1 cells are treated with Ad-Δ19 virus at a concentration of 1*10⁵ per chamber, and 540 represents capacitance change when HEP1 cells are treated with dl-ΔE1/ΔE3 virus at a concentration of 1*10⁵ per chamber.

In this regard, Ad-Δ19 virus is a virus having a death factor killing cells, dl-ΔE1 virus is a virus having a GFP (green fluorescent protein)-expressing protein therein, and dl-ΔE1/ΔE3 virus is an empty viral capsid.

At the concentration of 1*10⁵, the death factor of Ad-Δ19 virus does not greatly affect cell growth, and thus it is easy to perform comparison with dl-ΔE1 virus and dl-ΔE1/ΔE3 virus. After injection of three types of viruses, the capacitance increases to have a peak between 30 min to 50 min, and then immediately decreases. After the peak appears between 30 min to 50 min, capacitance gradually increases in the three types of viruses. The slope of the capacitance is similar to that of HEP1 cell during growth as a control, indicating that the cells grow well after the peak.

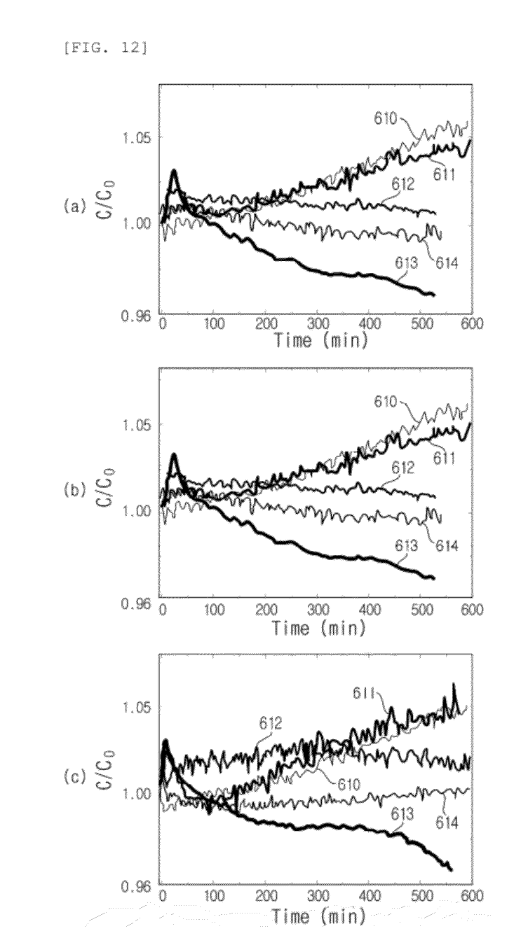

All three types of viruses have a peak, because the three types of viruses (Ad-Δ19 virus, dl-ΔE1 virus, dl-ΔE1/ΔE3 virus) are an adenovirus type and specific binding of its fiber knob with a CAR fiber receptor expressed on HEP1 cell membrane triggers internalization of the virus by endocytosis, leading to an increase in potential energy of the cell, as shown in FIG. 11. This result is also confirmed in the following experiments.

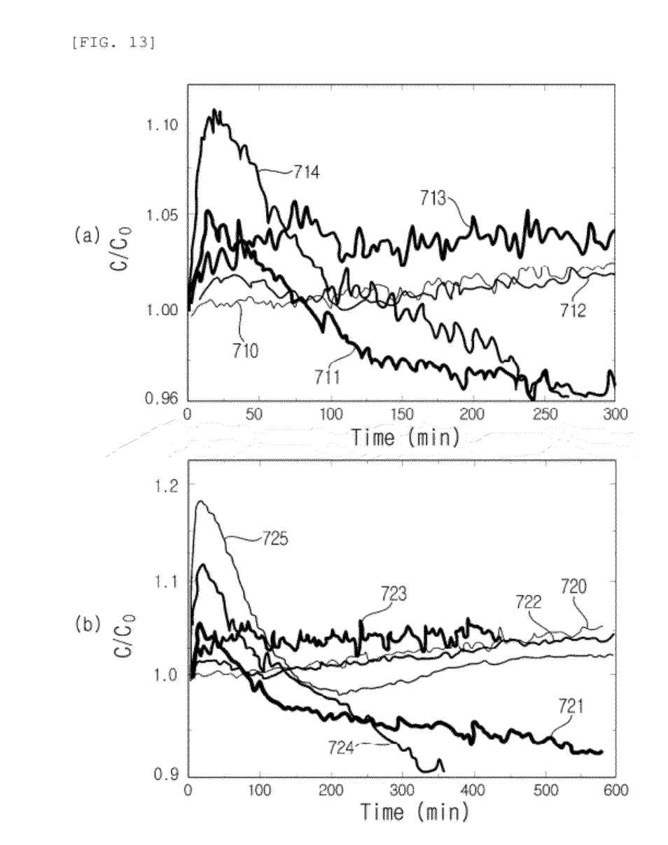

FIG. 12 is the result of real-time monitoring of cell capacitance when the cells are treated with (a) Ad-Δ19 virus, (b) dl-ΔE1 virus, and (c) dl-ΔE1/ΔE3 virus using a CAR-antibody specific to CAR, a protein inhibitor inhibiting all of the protein synthesis in the cell, and Ribavirin that is an antiviral agent to prevent viral replication in the cell by blocking viral RNA replication.

In (a) of FIG. 12, 610 represents capacitance change during HEP1 cell growth, 611 represents capacitance change when HEP1 cells are further treated with Δ19 virus having a death factor, 612 represents capacitance change when the cells are treated with the CAR-antibody and Δ19 virus, 613 represents capacitance change when the cells are treated with the protein inhibitor and Δ19 virus, and 614 represents capacitance change when the cells are treated with Ribavirin and Δ19 virus.

In (b), 621 represents capacitance change when the cells are treated with dl-ΔE1 virus having a GFP (green fluorescent protein)-expressing protein, 622 represents capacitance change when the cells are treated with dl-ΔE1 virus and CAR-antibody, 623 represents capacitance change when the cells are treated with the protein inhibitor and dl-ΔE1 virus, and 624 represents capacitance change when the cells are treated with Ribavirin and dl-ΔE1 virus.

In (c), 631 represents capacitance change when the cells are treated with dl-ΔE1/ΔE3 virus having only the viral capsid, 632 represents capacitance change when the cells are treated with dl-ΔE1/ΔE3 virus and CAR-antibody, 623 represents capacitance change when the cells are treated with the protein inhibitor and dl-ΔE1/ΔE3 virus, and 624 represents capacitance change when the cells are treated with Ribavirin and dl-ΔE1/ΔE3 virus.

In this experiment, the cells are treated with the CAR-antibody solution and then left for 20 min, followed by virus treatment, in order to sufficiently attach CAR-antibody to the cells. At this time, each of the Δ19 virus, dl-ΔE1 virus, and dl-ΔE1/ΔE3 virus was used at a concentration of 1*10⁵ per chamber, which does not affect the cell growth.

As a result, like in 611, 621, and 631, the initial peaks appear between 30˜50 min when the virus is only added, indicating that specific binding occurs. As shown in 612, 622, and 632, no initial peak appears between 30˜50 min when the virus is added together with CAR-antibody, and the capacitance gradually increases between 200˜300 min, and then decrease. No initial peaks appear because the CAR-antibody binds to the virus receptor (CAR) to inhibit endocytosis of the virus into the cell through the receptor. However, since the CAR-antibody binds to CAR and also internalized into the cell with time, the virus receptor of HEP1 cells are recovered to bind with the virus after a period of time, which is clarified even more by comparison with the addition of the protein inhibitor and Ribavirin.

That is, after internalization of CAR-antibody, the function of the virus receptor of HEP1 cells is recovered and thus endocytosis of the virus through the virus receptor gradually occurs at a delayed time point. The same results are also observed in Ad-Δ19 virus, dl-ΔE1 virus, and dl-ΔE1/ΔE3.

When endocytosis of the virus is observed after pretreatment of the cells with the protein inhibitor (613, 623, 633) and endocytosis of the virus is observed after pretreatment of the cells with Ribavirin (614, 624, 634), the initial peak appears unlike in the use of car-antibody, but the capacitance after the peak decreases due to the reduced cell function by inhibiting protein synthesis in the cells.

On the basis of the experimental results of using CAR-antibody that inhibits binding of the virus with the target receptor, it can be seen that the initial peak after the addition of the virus is attributed to endocytosis due to specific binding of the virus with the virus receptor present in the cell.

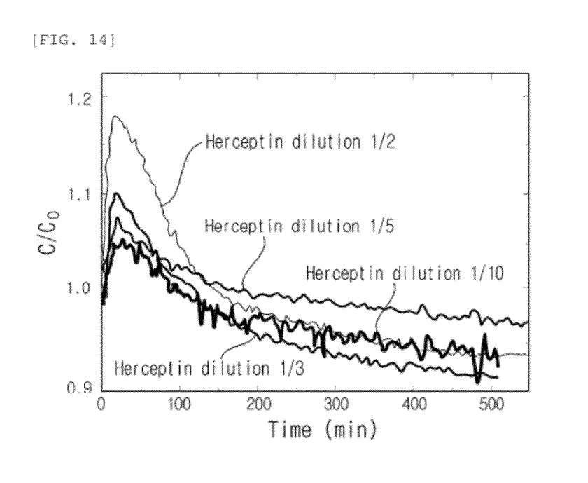

In FIG. 13, (a) is the result of real-time monitoring of cell capacitance when HEP1 cells are treated with Δ19 virus and CAR-antibody, and (b) is the result of real-time monitoring of cell capacitance when HEP1 cells are treated with Δ19 virus and CAR-antibody, and 435 breast cancer cells overexpressing ErbB-2 (HER2/neu) which is a target receptor of herceptin are treated with herceptin, for comparison therebetween.

In (a), 710 represents capacitance change during HEP1 cell growth, 711 represents capacitance change when the cells are treated with Δ19 virus of 1×10⁸ which is a concentration affecting cell growth, 712 represents capacitance change when the cells are treated with Δ19 virus of 1×10⁵ which is a concentration not affecting cell growth, 713 represents capacitance change when the cells are treated with CAR-antibody-1, and 714 represents capacitance change when the cells are treated with CAR-antibody-2. In (b), 720 represents capacitance change during HEP1 cell growth, 721 represents capacitance change when the cells are treated with Δ19 virus of 1×10⁸ which is a concentration affecting cell growth, 722 represents capacitance change when the cells are treated with Δ19 virus of 1×10⁵ which is a concentration not affecting cell growth, 713 represents capacitance change when the cells are treated with CAR-antibody-1, 714 represents capacitance change when the cells are treated with CAR-antibody-2, and 725 represents capacitance change when 435 breast cancer cells overexpressing ErbB-2 (HER2/neu) are treated with herceptin

As shown in (a) of FIG. 13, the results of measuring capacitance while HEP1 cells are treated with the CAR receptor-specific CAR-antibody in the same manner as in the treatment of adenovirus show that the initial peak is attributed to endocytosis due to specific binding of the biomolecule with its target receptor. It was found that the peaks also appear around the same time as in the virus treatment, even when the CAR-antibody is used.

As shown in (b) of FIG. 13, the results of measuring capacitance while 435 breast cancer cells overexpressing ErbB-2 (HER2/neu) receptor are treated with herceptin targeting the ErbB-2 (HER2/neu) receptor as a monoclonal antibody drug in the same manner show that the peak similar to those in the virus and CAR-antibody treatment is observed. Therefore, the results of adenovirus, CAR-antibody, and Herceptin treatment suggest that the cell sensor for real-time monitoring of cell capacitance according to an exemplary embodiment of the present invention is able to measure binding of the biomolecule with the cell receptor by measurement of capacitance.

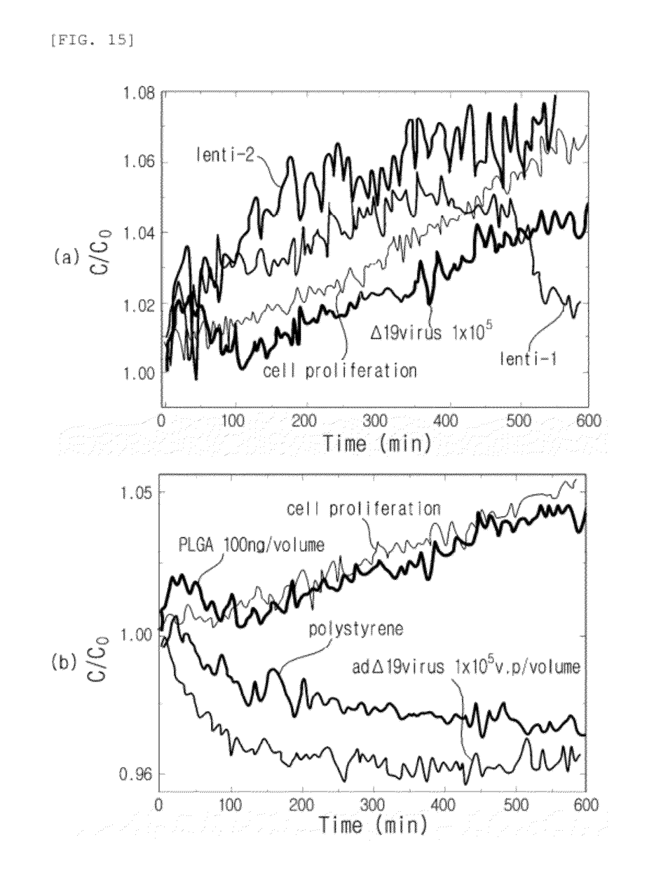

FIG. 14 is the result of real-time monitoring of cell capacitance when 435 breast cancer cells are treated with different concentrations of herceptin.

As illustrated, herceptin specifically binds to the ErbB-2 (HER2/neu) receptor expressed in 435 breast cancer cells, and therefore the initial peaks differ depending on the concentrations of herceptin.

FIG. 15 is the result of real-time monitoring of cell capacitance when endocytosis by adsorption occurs.

As illustrated in (a) of FIG. 15, when the cells are treated with a type of retrovirus, lentivirus which is internalized via endocytosis by adsorption not via endocytosis through the target receptor, no initial peak is observed unlike in the endocytosis through the target receptor.

As illustrated in (b) of FIG. 15, when the cells are treated with each of polystyrene beads and PLGA (polylactic-co-glycolic acid) particles that are internalized by adsorption, no initial peak is observed unlike in the treatment of the virus, but like in the treatment of lentivirus.

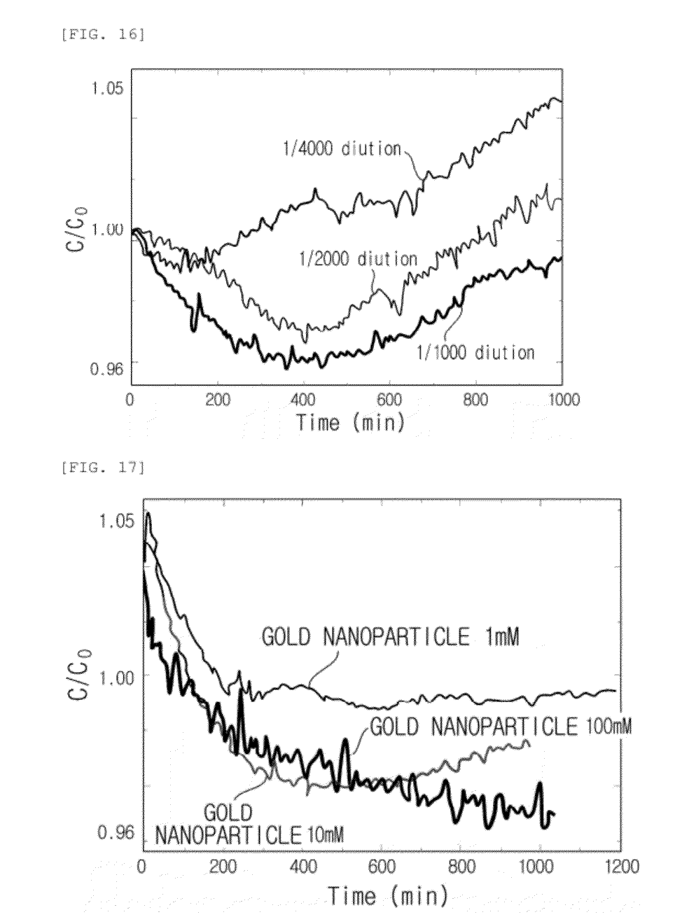

This result suggests that the increased potential energy due to endocytosis is observed only during the endocytosis by specific binding of the target receptor, and is not observed during the endocytosis by adsorption. This result is clarified even more by FIG. 16.

FIG. 16 is the result of real-time monitoring of cell capacitance when the cells are treated with different concentrations of polystyrene beads.

No initial peak is observed in the treatment of polystyrene beads that are internalized without specific binding of the receptor. In the concentration-dependent capacitance, endocytosis by adsorption of beads onto cells can be identified by the decrease of capacitance, and the decrease of capacitance occurs more quickly, as the concentration is lowered.

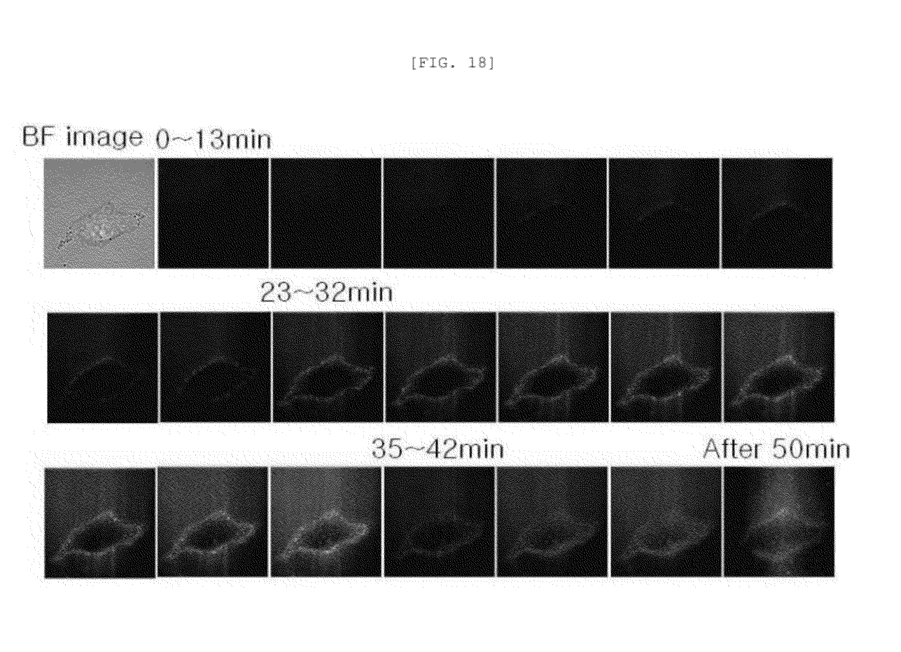

FIG. 17 is the result of real-time monitoring of cell capacitance when the cells are treated with different concentrations of gold nanoparticles.

No initial peak is also observed in the treatment of gold nanoparticles that are internalized by adsorption without specific binding of the receptor. The internalization of gold nanoparticles into cells can be identified by the decrease of capacitance, and the decrease of capacitance occurs more quickly, as the concentration is lowered.

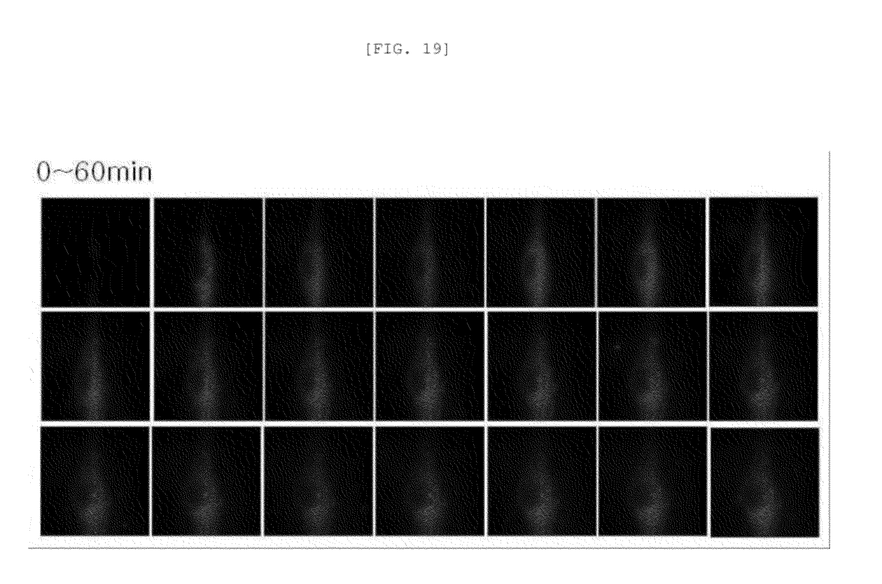

FIG. 18 is TIRF (Total Internal Reflection Fluorescence) microscope images showing the endocytosis of GFP-tagged virus via the receptor on the cell.

As illustrated, the virus is visualized as a green fluorescence spot, and a stronger green emission is observed on the surface of the cell with time. After the strongest green emission is observed in the image corresponding to 32 min, it becomes weaker. That is, the virus binds to the target receptor (CAR) developed on the cell membrane for endocytosis until 32 min. After 32 min, the virus is internalized into the cells, and therefore the green emission becomes weaker. The strongest green emission in TIRF images and the initial peak in LCR measurement are observed at a similar time point, indicating that the peak appears upon specific binding of the biomolecule to the cell receptor.

FIG. 19 is TIRF (Total Internal Reflection Fluorescence) microscope images showing the endocytosis of rhodamine (red dye)-loaded polystyrene bead by adsorption.

As illustrated, the red emission corresponding to the bead is persistently observed, indicating that it is not endocytosis by a target receptor but endocytosis by adsorption.

Taken together, the method for real-time monitoring of cell capacitance using the cell sensor according to an exemplary embodiment of the present invention is able to monitor the endocytosis of biomolecules in real time using the capacitance sensor, which had been hardly captured by general fluorescence microscopy. According to the method for real-time monitoring of cell capacitance using the cell sensor, a peak appears due to potential change of the cell upon endocytosis of biomolecule through the target receptor (CAR). As an experimental target used in the monitoring method, the biomolecule includes adenovirus, CAR-antibody, and Herceptin as well as a variety of viruses used for gene delivery, and thus biological mechanisms can be analyzed by electric signals, thereby providing a valuable tool for basic research.

The above description have been disclosed for illustration of the technical spirit of the present invention, and one of ordinary skill in the art, to which this invention belongs, will appreciate that various modifications, additions and substitutions are possible, without departing from the scope and spirit of the invention. It should be understood that the embodiments and the accompanying drawings disclosed in the present invention have been described for illustrative but not limitative purposes, and the scope of the present invention is not intended to be limited by these embodiments and accompanying drawings. The scope of the invention should be determined with references to the appended claims and includes the full scope of equivalents to which such claims are entitled.

1. A cell sensor for real-time monitoring of cell capacitance, comprising: a substrate; a first electrode and a second electrode that are formed on the substrate and spaced apart by a gap from each other, in which at least one or more cells are introduced to be attached onto the gap; and a passivation layer that is formed on each of the tops of the first electrode and the second electrode to prevent the cells from being attached onto the top of the first electrode and the second electrode.

2. The cell sensor of claim 1, wherein the first electrode and the second electrode includes any one material of gold, platinum, and conductive polymers.

3. The cell sensor of claim 1, wherein the capacitance between the first electrode and the second electrode is measured to monitor the cell condition in real time.

4. The cell sensor of claim 1, wherein the gap size between the passivation layers formed on each of the tops of the first electrode and the second electrode is identical to that between the first electrode and the second electrode.

5. The cell sensor of claim 1, wherein the passivation layer is formed using a non-conductive material.

6. The cell sensor of claim 1, wherein the passivation layer includes one or more of PMMA (Polymethyl Methacrylate) and silicon oxide (SiO₂).

7. The cell sensor of claim 1, further comprising a fluidic channel that is formed to have a gap area and includes an inlet to introduce one or more of biomolecules, which undergo an antigen-antibody reaction with the cells, and cell death-inducing agents into the gap area and an outlet to extract one or more of biomolecules and cell death-inducing agents from the gap area.

8. The cell sensor of claim 7, wherein the fluidic channel include one or more of acryl and PDMS (Polydimethylsiloxane).

9. The cell sensor of claim 7, wherein the fluidic channel is attachable and detachable to and from the substrate and the first and second electrodes.

10. The cell sensor of claim 1, further comprising a well to prevent a cell culture medium from leaking out of the gap area.

11. The cell sensor of claim 10, wherein the well include one or more of acryl and PDMS (Polydimethylsiloxane).

12. The cell sensor of claim 1, comprising a plurality of electrode pairs composed of the first and second electrodes, which are arrayed on the substrate in common.

13. A method for monitoring a cell condition in real time, comprising the steps of: (a) introducing the cells having target receptors into the gap area of the cell sensor of any one of claims 1 to 12 and then introducing a cell culture medium for cell cultivation thereto; (b) introducing a biomolecule capable of specifically binding with the target receptors of the cells cultured in the cell culture medium; and (c) measuring capacitance between the first electrode and the second electrode in real time during cultivation of the cells of step (b).

14. The method of claim 13, wherein the cell is any one of HEP1 cell highly expressing CAR (coxsackie and adenovirus receptor) which is a target receptor of adenovirus and CAR-antibody, and 435 breast cancer cell overexpressing ErbB-2 (HER2/neu) which is a target receptor of herceptin.

15. The method of claim 13, wherein the cell culture medium includes any one of DMEM (Dulbecco's Modified Eagle Medium), RPMI (Roswell Park Memorial Institute) 1640 medium, and MEM (Minimum Essential Medium).

16. The method of claim 13, wherein the biomolecule is any one of adenovirus, CAR-antibody, and herceptin.

17. The method of claim 13, wherein in step (c), endocytosis of the biomolecules into the cells through the cell surface receptor can be monitored in real time by real-time measurement of capacitance.

18. The method of claim 13, further comprising the step of controlling the concentration of the biomolecule using the cell culture medium, prior to step (c).

19. A method for monitoring a cell condition in real time, comprising the steps of: (a) introducing the cells having target receptors into the gap area of the cell sensor of any one of claims 1 to 12 and then introducing a cell culture medium for cell cultivation thereto; (b) introducing a metal particle or a cell-death inducing agent into the cells cultured in the cell culture medium; and (c) measuring capacitance between the first electrode and the second electrode in real time during cultivation of the cells of step (b).

20. The method of claim 19, wherein the cell-death inducing agent includes any one of viruses, bacteria, nucleic acids, and drugs that contain TRAIL (Tumor necrosis factor Related Apoptosis Inducing Ligand).

21. The method of claim 19, wherein in step (c), endocytosis of the cell-death inducing agent into the cells through the cell surface receptor or adsorption can be monitored in real time by real-time measurement of capacitance.

22. The method of claim 19, further comprising the step of controlling the concentration of the cell-death inducing agent or the metal particles using the cell culture medium, prior to step (c).

23. A method for monitoring a cell condition in real time, comprising the steps of: (a) introducing the cells having target receptors into the gap area of the cell sensor of any one of claims 1 to 12 and then introducing a cell culture medium for cell cultivation thereto; (b) introducing a biomolecule capable of specifically binding with the target receptors of the cells cultured in the cell culture medium, together with a cell-death inducing agent; and (c) measuring capacitance between the first electrode and the second electrode in real time during cultivation of the cells of step (b).

24. The method of claim 23, further comprising the step of controlling the concentration of any one of the biomolecule and the cell-death inducing agent using the cell culture medium, prior to step (c).
